(19) United States
(12) Patent Application Publication
(10) Pub. No.: US 2007/0141726 A1
(43) Pub. Date: Jun. 21, 2007

(54) Detection via switchable emission of nanocrystals

(75) Inventors: Jackie Y. Ying, Singapore (SG); Hsiao-Hua Yu, Cavendish Park (SG); Emril Mohamed Ali, Neptune Court (SG); Nikhil R. Jana, West Bay (SG)

Correspondence Address: WOLF GREENFIELD & SACKS, P.C., 600 ATLANTIC AVENUE, BOSTON, MA, 02210-2206, US

(73) Assignee: Agency for Science, Technology and Research, Centros (SG)

(21) Appl. No.: 11/313,218

(22) Filed: Dec. 19, 2005

(51) Int. Cl. G01N 33/553

(52) U.S. Cl. 436/525

(57) ABSTRACT

The present invention relates to methods for determination of an analyte. The invention provides various methods involving exposure of a luminescent material to an analyte wherein, upon interaction with the analyte, a change in luminescence may be observed as a function of the duration of exposure to electromagnetic radiation, thereby determining the analyte. Some embodiments of the invention include the use of highly emissive semiconductor nanocrystals.

### FIELD OF THE INVENTION

The present invention relates to methods for determining an analyte using luminescent articles.

### BACKGROUND OF THE INVENTION

This invention is relevant to articles that are emissive of electromagnetic radiation (such as visible light). One category of such articles is semiconductor nanocrystals, or quantum dots, are highly emissive materials that may be particularly useful in a variety of applications. For example, semiconductor nanocrystals may have narrow and highly symmetric emission spectra, making them attractive for use as diagnostic tools, such as fluorescent probes in biological labeling and diagnostics. In some cases, semiconductor nanocrystals have been employed in fluorescence resonance energy transfer and fluorescence quenching assays. Semiconductor nanocrystals may also exhibit high emission stability over long periods of time, providing advantages over conventional biological probing dyes.

Due to quantum confinement effects, many semiconductor nanocrystals may exhibit size-dependent optical properties. That is, the wavelength at which the semiconductor nanocrystal emits light may depend on the size of the nanocrystal, and the emission wavelength may be controlled by controlling the particle diameter. For example, one excitation wavelength may be used to excite a population of semiconductor nanocrystals having different sizes, resulting in the emission of many different wavelengths of light due to the excitation wavelength. This makes semiconductor nanocrystals quite useful in many settings.

### SUMMARY OF THE INVENTION

The present invention provides methods for determining an analyte via interaction of the analyte with a luminescent article, the methods comprising in certain embodiments providing a sample suspected of containing an analyte, exposing the sample to a luminescent article comprising an outer layer, and, if the analyte is present, allowing the analyte to become immobilized with respect to the article via interaction between the analyte and the outer layer, wherein the outer layer is modified by said interaction; determining a first emission of the luminescent article; exposing the nanoparticle to electromagnetic radiation for a period of time and under conditions sufficient to cause a change in a luminescence characteristic of the nanoparticle; determining a second emission of the luminescent article; and determining a variance between the first emission and the second emission indicative of the presence of the analyte, wherein modification of the outer layer increases the susceptibility of the article to a change in the luminescence characteristic upon exposure of the article to the electromagnetic radiation for the period of time and under the conditions, such that, in the absence of the analyte, exposure of the luminescent article to the electromagnetic radiation for the period of time and under the conditions does not result in said variance between the first and second emissions.

The present invention also provides methods for determining an analyte, the methods of certain embodiments comprising exposing a luminescent article to electromagnetic radiation in the presence of a sample suspected of containing an analyte, wherein the analyte affects a change in a luminescence characteristic of the article responsive to the electromagnetic radiation; and if the analyte is present, determining the analyte by determining a change in the luminescence characteristic of the article resulting from said exposure to electromagnetic radiation.

### BRIEF DESCRIPTION OF THE DRAWINGS

FIG. 1 illustrates a nanoparticle comprising an outer layer and a core according to one embodiment of the present invention.

FIG. 2 shows the reversible reaction between amine-functionalized ZnO nanocrystals and aldehydes, according to one embodiment of the present invention.

FIG. 3 shows the (a) absorption and (b) emission spectra of an amine-functionalized ZnO nanocrystals according to one embodiment of the present invention.

FIG. 4 shows the kinetic measurements of an illustrative embodiment of an amine-functionalized ZnO nanocrystal in the absence and presence of aldehydes in either (a) water or (b) a 5 mM borate buffer solution.

FIG. 5 shows the emission spectra of amine-functionalized ZnO nanocrystals in the presence of (a) 0.05 mM, (b) 0.125 mM, and (c) 0.25 mM of o-phthaldehyde, according to one embodiment of the invention.

FIG. 6 shows TEM images of amine-functionalized ZnO nanocrystals (a) before and (b) after treatment with 0.05 mM of o-phthaldehyde under UV irradiation for 10 minutes.

FIG. 7 shows the luminescence intensity of amine-functionalized ZnO nanocrystals in the presence of various aldehydes after two minutes of UV irradiation.

FIG. 8 shows the luminescence intensity of amine-functionalized ZnO nanocrystals in the presence of various organic analytes after two minutes of UV irradiation.

### DETAILED DESCRIPTION

The present invention relates to methods for determination of an analyte. The invention provides various methods involving exposure of an emissive material, such as a luminescent material to an analyte wherein, upon interaction with the analyte, a change in luminescence may be observed as a function of the duration of exposure to electromagnetic radiation, whereby the presence and/or amount of analyte can be determined. Some advantages of the invention include the use of highly emissive articles, as well as a simplified, method for direct determination of biological molecules, involving only two components.

In one aspect, the invention involves the appreciation that a species can bind to an article and, thereby, change the article's susceptibility to electromagnetic radiation. I.e., a species can be made to bind to an article, whereupon exposure of the article to electromagnetic radiation changes an emissive property of the article (where the emissive property would not be changed, or at least not to the same degree, were the species not present). This leads to use of this phenomenon for detection of the species as an analyte. In some embodiments, the method may comprise the exposure of a luminescent article to electromagnetic radiation in the presence of a sample suspected of containing an analyte, wherein, if the analyte is present, the analyte affects a change in a luminescence characteristic of the article responsive to the electromagnetic radiation. For example, the luminescence characteristic may be the intensity, wavelength, or occurrence of fluorescence emission. The analyte may be determined by determining the change in the luminescence characteristic of the article upon electromagnetic radiation. In some cases, the change in the luminescence characteristic upon electromagnetic radiation in the absence of the analyte may be different (e.g., smaller in magnitude) from the change in the luminescence characteristic in the presence of the analyte upon electromagnetic radiation.

As used herein, the term “determining” generally refers to the analysis of a species or signal, for example, quantitatively or qualitatively, and/or the detection of the presence or absence of the species or signals. “Determining” may also refer to the analysis of an interaction between two or more species or signals, for example, quantitatively or qualitatively, and/or by detecting the presence or absence of the interaction. The term “nanoparticle” generally refers to a particle having a maximum cross-sectional dimension of no more than 1 μm. Nanoparticles can be made of material that is, e.g., inorganic or organic, polymeric, ceramic, semiconductor, metallic, non-metallic, crystalline (e.g., “nanocrystals”), amorphous, or a combination. Typically, nanoparticles are of less than 250 nm cross section in any dimension, more typically less than 100 nm cross section in any dimension, and preferably less than 50 nm cross section in any dimension. In some embodiments, the nanoparticles may have a diameter of about 2 to about 50 nm. In some embodiments, the nanoparticles may have a diameter of about 2 to about 20 nm. In further embodiments, the nanoparticles may have diameters of about 2 to about 3 nanometers.

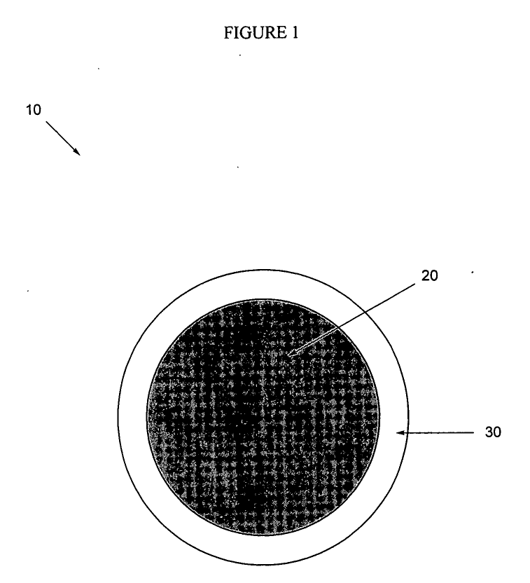

In some embodiments, the present invention provides methods for determining an analyte via interaction of the analyte with a luminescent article. Luminescent articles used in the present invention may have a luminescent core at least partially covered by an outer layer to which the analyte is exposed (e.g., the article can be a particle at least partially encapsulated by the outer layer). As an example shown in FIG. 1, luminescent article 10 comprises a luminescent core 20 and an outer layer 30, which surrounds luminescent core 20. Various luminescence characteristics (e.g., emission intensity, emission wavelength, and the like) associated with the article may depend on the protective, outer layer surrounding the article. The outer layer may be composed of a material appropriately chosen to be, for example, electronically insulating (e.g., through augmented redox properties), optically non-interfering, chemically stable, or lattice-matched to the underlying material (e.g., for epitaxial growth, minimization of defects). The outer layer may comprise an inorganic material, an organic material, or combinations thereof, as described more fully below. In some embodiments, the presence of the outer layer may provide chemical and photochemical stability to a luminescent core upon, for example, exposure to electromagnetic radiation (e.g., UV light). In some embodiments, interaction of the outer layer with the analyte may cause a disruption in the structure of the outer layer, including lattice distortion, crystal dissolution, or other deformations, for example.

Methods described herein may comprise exposing a sample suspected of containing an analyte to a luminescent article comprising a luminescent core at least partially encapsulated by an outer layer. If the analyte is present, the analyte may become immobilized with respect to the article via interaction between the analyte and the outer layer. In some cases, the interaction may involve modification of the outer layer such that the susceptibility of the luminescent article to a change in the luminescence characteristic may be increased upon exposure of the article to the electromagnetic radiation. Upon interaction between the analyte and the outer layer, the luminescent article may be exposed to electromagnetic radiation and a first emission may be determined. Subsequently, upon exposure of the nanoparticle to electromagnetic radiation for a period of time and under conditions sufficient to cause a change in a luminescence characteristic of the nanoparticle, a second emission of the luminescent article may be determined. The variance between the first emission and the second emission may indicate the presence of the analyte.

In some cases, exposure of the luminescent article to electromagnetic radiation in the absence of the analyte may cause a slight change in the emission of the article. The change in the luminescence characteristic may be attributed to, for example, interface defects between the emissive core and the outer layer, surface imperfections or “traps” that enhance nonradiative deactivation pathways (or inefficient radiative pathways), the gross morphologies of the particle, the presence of impurities, or the like. However, any change or variance in emission observed in the absence of analyte may be different when compared to the change in emission that may occur in the presence of analyte. In some embodiments, the variance between the first and second emissions that occurs in the presence of analyte may be significantly larger than the variance between the first and second emissions that occurs in the absence of analyte.

In some embodiments, the outer layer may interact with an analyte to form a bond with the analyte, such as a covalent bond (e.g. carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), a dative bond (e.g. complexation or chelation between metal ions and monodentate or multidentate ligands), or the like. The interaction may also comprise Van der Waals interactions. In one embodiment, the interaction comprises forming a covalent bond with an analyte. The outer layer may also interact with an analyte via a binding event between pairs of biological molecules. For example, the outer layer may comprise an entity, such as biotin that specifically binds to a complementary entity, such as avidin or streptavidin, on a target analyte.

In some cases, the outer layer may be a self-assembled, tightly-packed structure and, in the presence of the analyte, the outer layer may interact with (e.g., form a bond with) the analyte to disrupt the self-assembled, tightly-packed structure, increasing the susceptibility of the article to the change in the luminescence characteristic upon exposure of the article to the electromagnetic radiation. In some cases, without an adequate (e.g, tightly-packed) protective outer layer, the luminescence intensity of the luminescent article may be reduced upon exposure to electromagnetic radiation and may undergo photobleaching. The term “photobleaching” is known in the art and refers to a reduction in luminescence intensity upon exposure to electromagnetic radiation, where the degree of reduction may be a function of the duration of exposure to electromagnetic radiation. Photobleaching may cause a material to substantially lose its ability to emit light upon exposure to electromagnetic radiation. In some embodiments, such photobleaching may be due to lattice distortion or crystal dissolution of the luminescent article.

In some embodiments, the outer layer of the luminescent article may comprise a plurality of functional groups having an affinity for a surface of the article. However, in the presence of analyte, the analyte may become immobilized with respect to the article, causing the functional groups to become increased in separation from the surface of the article, increasing the susceptibility of the article to a change in a luminescence characteristic upon exposure of the article to the electromagnetic radiation (i.e., for a defined period of time). For example the functional group may be converted to a sterically large group upon interaction with an analyte, such as a protein. The sterically large groups may prevent the formation of a tightly-packed outer layer. In some embodiments, the affinity of the functional group for the surface of the article may by altered (e.g., decreased) upon interaction with the analyte. The functional group may also become converted into a different functional group upon interaction with the analyte. In one embodiment, the article comprises amine groups, wherein, upon interaction with an analyte comprising aldehyde groups, the amine is converted to an imine, which may have a decreased affinity for the surface of the article, relative to the amine.

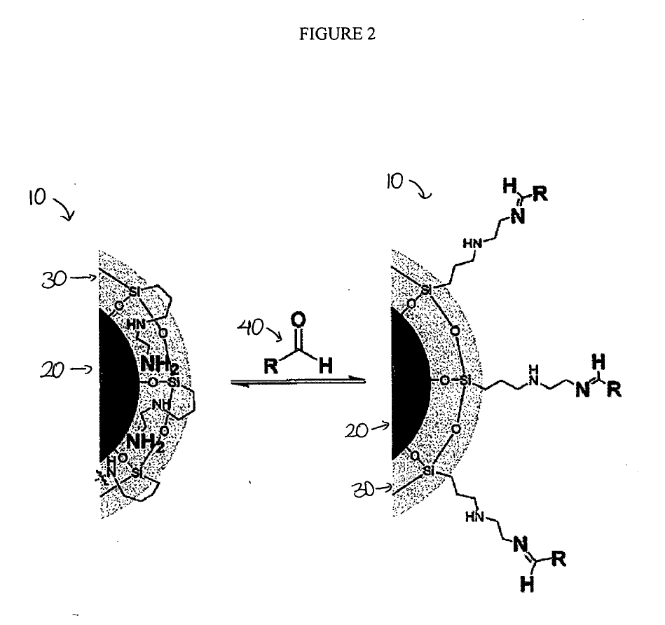

In the illustrative embodiment shown in FIG. 2, luminescent article 10 comprises a luminescent core 20 and an outer layer 30, which is a tightly-packed structure at the surface of the nanoparticle. Luminescent core 20 may comprise a semiconductor nanocrystal or a fluorescent dye, for example. In one embodiment, luminescent core 20 comprises ZnO. The outer layer 30 comprises heteroalkyl chains having terminal amine groups, which may react with aldehydes reversibly to form imines. Exposure of luminescent article 10 to an aldehyde-substituted analyte 40 may result in the formation of a covalent bond between luminescent article 10 and aldehyde-substituted analyte 40 via imine formation, causing the outer layer 30 to become dispersed from the surface of the nanoparticle. That is, the heteroalkyl chains become elongated such that the imine moiety is increased in separation from the surface. In some cases, this may be due to a change in affinity of the outer layer for the surface of the nanoparticle. For example, in the illustrative embodiment, the amine functional group may have an affinity for the surface of the nanoparticle but upon reaction with the analyte to form an imine, has a decreased affinity for the surface of the nanoparticle. In some cases, the outer layer may become dispersed, for example, by the elongation of alkyl or heteroalkyl chains due to the size of the analyte bonded to the outer layer. For example, the analyte may be a sterically bulky analyte, such as a protein or other biological analyte, which may prevent formation of a tightly-packed outer layer. The dissolution of the tightly-packed structure of the outer layer may result in the loss of photostability and occurrence of photobleaching upon exposure to electromagnetic radiation (e.g., UV, visible, IR, etc.), indicating the presence or amount of the analyte.

In some embodiments, alteration of the protective outer layer may also cause the luminescent article to become more susceptible to dissolution of the luminescent core. A luminescent article may have a certain diameter (e.g., grain size) that may be reduced upon exposure to electromagnetic radiation in the presence of an analyte, resulting from increasing photodissolution of the luminescent article upon interaction of the outer layer with the analyte. For example, in one embodiment, a luminescent article may comprise a semiconductor nanocrystal having a first diameter, which, upon disruption of the protective outer layer by the analyte, may decompose to a semiconductor nanocrystal having a smaller, second diameter upon exposure to electromagnetic radiation.

Methods of the present invention may be distinguished from other methods, where a decrease in luminescence may be observed due to luminescence quenching. In such cases, “quenching” occurs when a chromophore in an excited state is exposed to an “acceptor” species that can absorb energy from the excited state chromophore, which then returns to a ground state due to nonradiative processes (i.e. without emitting radiation), resulting in a reduced quantum yield (e.g., number of photons emitted per adsorbed photon). In contrast, embodiments of the present invention may involve a change in the luminescence decay (e.g., photobleaching) of an emissive material to determine the analyte. The degree of luminescence decay may be based on the duration of exposure to electromagnetic radiation.

Advantages of methods of the present invention include a simple, two-component method for signal transduction using luminescent nanoparticles. Unlike more complex assays involving multiple components, the present invention involves direct bonding of the luminescent nanoparticle to the analyte for determination of the analyte. Methods of the invention may be broadly applicable to biological and chemical sensors and assays. For example, the nanoparticles may be easily functionalized with a wide variety of biological or chemical moieties to suit a particular application. Methods of the present invention may also be highly sensitive (e.g., for less than <1 mM analyte) and selective for an analyte, as described more fully in the examples below.

In some embodiments, the analyte may be a chemical or biological analyte. The term “analyte,” may refer to any chemical, biochemical, or biological entity (e.g. a molecule) to be analyzed. In some cases, luminescent articles of the present invention may have high specificity for the analyte, and may be a chemical, biological, or explosives sensor, for example. In some embodiments, the analyte comprises a functional group that is capable of interacting with at least a portion of the luminescent article. For example, the functional group may interact with the outer layer of the article by forming a bond, such as a covalent bond. Some embodiments involve analytes that comprise an aldehyde. In one set of embodiments, the analyte may interact with a functional group within the outer layer such that the analyte pulls the functional group away from the surface of the luminescent article.

As described herein, the luminescent article comprises an outer layer or shell that encapsulates, or partially encapsulates, a luminescent core. In some embodiments, it is preferable for the outer layer to encapsulate the majority of the surface area of the emissive core. For example, the outer layer may encapsulate at least 75% of the surface area of the core. In some cases, the outer layer may completely encapsulate the emissive core. In some embodiments, the outer layer is not chemically bound to the emissive core (e.g., quantum dot, fluorescent dye, other fluorescent material) and yet may contain the luminescent article by encapsulation. Thus, the outer layer and emissive core may be devoid of ionic bonds and/or covalent bonds and/or dative bonds between them. In some cases, the outer layer may comprise an organic material (e.g., based on carbon and/or polymers of carbon). In some cases, the outer layer may comprise a non-organic material (e.g., not based on carbon and/or polymers of carbon, but nonetheless may include carbon atom). It may be preferred for the outer layer to be non-organic and may be formed of a silicon polymer such as silica. In certain embodiments, the outer layer may be porous. For example, the outer layer may have pores on the mesoscale size. In certain embodiments, the outer layer may be non-porous.

In some embodiments, the outer layer may be appropriately functionalized to impart desired characteristics (e.g., surface properties) to the luminescent article. For example, the outer layer may be functionalized or derivatized to include compounds, functional groups, atoms, or materials that can alter or improve properties of the luminescent article. In some embodiments, the outer layer may comprise functional groups which can specifically interact with an analyte to form a covalent bond. In some embodiments, the outer layer may include compounds, atoms, or materials that can alter or improve properties such as compatibility with a suspension medium (e.g., water solubility, water stability), photo-stability, and biocompatibility. In some cases, the outer layer may comprise functional groups selected to possess an affinity for the surface. In some cases, the outer layer may comprise functional groups which possess an affinity for the surface, wherein the functional group may be altered (e.g., chemically) such that its affinity for the surface is altered (e.g., decreased).

In some embodiments, the outer layer comprises a material which improves the luminescent (e.g., fluorescent) properties of the luminescent article. For example, the outer layer may comprise a material (e.g., a passivation material) that may eliminate energy levels at the surface of the crystal that may act as traps for electrons and holes that degrade the luminescent properties of the quantum dot. That is, the outer layer may comprise materials which prevent photobleaching of the emissive core. In some embodiments, the passivation material may be non-conductive and/or non-semiconductive. For example, the passivation material may not exhibit a higher band gap than a nanocrystal which it surrounds. In specific embodiments, the passivation material may be non-ionic and non-metallic. A non-conductive material is a material that does not transport electrons when an electric potential is applied across the material. The passivation material can be comprised of, or consist essentially of, a compound exhibiting a nitrogen-containing functional group, such as an amine. The amine may be bound directly or indirectly to one or more silicon atoms such as those present in a silane or other silicon polymer. The silanes may include any additional functional group such as, for example, alkyl groups, hydroxyl groups, sulfur-containing groups, or nitrogen-containing groups. Examples of passivation material include amino silanes such as amino propyl trimethoxysilane (APS) can be used. The use of APS in quantum dots has been shown to provide passivation and to improve quantum yields to a level comparable to the improvements obtained by the use of higher band gap passivation layers such as those made of zinc sulfide (ZnS).

The outer layer may also comprise functional groups capable of binding an analyte (e.g., via formation of a bond, via interaction between pairs of biological molecules, etc.). In one embodiment, the functional group may be positioned in close enough proximity to the luminescent core physically, or within sufficient electronic, inductive, or steric communication range of the luminescent core, such that interaction of the analyte with the functional group causes a detectable change in a luminescent characteristic of the luminescent article. In some cases, the functional group may form a bond with an analyte. The functional group may comprise an “electrophilic” atom, which refers to an atom which may be attacked by, and forms a new bond to, a nucleophile. In some cases, the electrophilic atom may comprise a suitable leaving group. The functional group may also be “nucleophilic” and may have a reactive pair of electrons. For example, the outer layer may comprise a carbonyl group such as an aldehyde, an ester, a carboxylic acid, a ketone, an amide, an anhydride, or an acid chloride, a thiol, a hydroxyl group, an amine, a cyano group, charged moieties, or the like. In some embodiments, the outer layer comprises an amine, a thiol, a carboxylic acid, an anhydride, or an alcohol. In some embodiments, the outer layer comprises an amine. In some cases, the functional group (e.g., an amine) may be attached to the surface of the outer layer via an alkyl or heteroalkyl chain.

The outer layer may also comprise a functional group that acts as a binding site for an analyte. The binding site may comprise a biological or a chemical molecule able to bind to another biological or chemical molecule in a medium, e.g. in solution. For example, the binding site may be capable of biologically binding an analyte via an interaction that occurs between pairs of biological molecules including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. In some cases, the luminescent articles may be used in applications such as drug discovery, the isolation or purification of certain compounds, or high-throughput screening techniques.

Functional groups may be selected to suit a particular application. In some cases, functional groups may be chosen based on their affinity for the surface of the luminescent article (e.g., the outer layer). For example, a functional group at the terminal end of an alkyl or heteroalkyl chain may be chosen for its affinity for the outer layer and its ability to cluster at the surface of the article and form a tightly-packed structure. In some cases, the functional group may be selected based on the ability to have an interaction with a particular analyte. One screening test for selection of an appropriate functional group may involve placing the functionalized article in solution with an analyte and evaluating the ability of the functional group to bind the analyte (e.g., via formation of a covalent bond or via interaction between pairs of biological molecules). Additionally, the ability of the functional group to be pulled away from the surface of the article by the analyte to effect a sufficient change in a luminescent characteristic of the article may be evaluated (i.e., by monitoring the degree to which the luminescence of the article decreases as a function of exposure to electromagnetic radiation in the presence of the analyte).

In some embodiments, a hydrophilic species may be associated with the outer layer (e.g., a silica outer layer) to provide greater hydrophilicity to the composite. The hydrophilic species can be, for example, amines, thiols, alcohols, carboxylic acids and carboxylates, sulfates, phosphates, a polyethylene glycol (PEG) or a derivative of polyethylene glycol. Derivatives include, but are not limited to, functionalized PEGs, such as amine, thiol and carboxyl functionalized PEG. The hydrophilic species can be chemically bound to the outer layer or can be, for example, physically trapped by the outer layer material. Preferably, the hydrophilic species includes a portion that can be chemically bonded to the outer layer and a second portion that provides hydrophilicity and may extend outwardly from the surface of the outer layer.

Presence of such glycols can impart superior water solubility characteristics to the composites while being biocompatible and nontoxic and can, in some instances, provide for better dispersion of the luminescent articles in solution. For example, by integrating PEG into the outer layer (which may be silica), the composite may be rendered water soluble at pHs of less than 8.0 or less than or equal to 7.0. Thus, these composites may be water soluble at neutral or below neutral pHs and thus may be biocompatible and appropriate for use in biological fluids such as blood and in vivo. In some embodiments, the inclusion of PEG into the silica outer layer can enable the composites to remain in solution for extended time periods (e.g., greater than 6 hours). The term “water soluble” is used herein as it is commonly used in the art to refer to the dispersion of a luminescent article in an aqueous environment. “Water soluble” does not mean, for instance, that each material is dispersed at a molecular level. A luminescent article can be composed of several different materials and still be “water soluble” as an integral particle. In addition, the presence of PEG or related compounds in the silica outer layer can provide for a material exhibiting a reduced propensity to adsorb protein, cells, and other biological materials. This means that, for example, when used in vivo, the composites can stay in solution for a longer period of time than do similar composites, thus allowing for increased circulation and improved deliverability to intended targets.

The outer layer may have a thickness great enough to encapsulate the core to the extent desired. In some embodiments, the outer layer may have an average thickness of less than 50 nanometers; and, in some embodiments, the outer layer may have an average thickness of less than 25 nanometers (e.g., between 5 nanometers and 20 nanometers). The average outer layer thickness may be determined using standard techniques by measuring the thickness at a representative number of locations using microscopy techniques (e.g., TEM). Examples of suitable outer layer materials include, but are not limited to, polystyrene, polyacrylate, or other polymers, such as polyimide, polyacrylamide, polyethylene, polyvinyl, poly-diacetylene, polyphenylene-vinylene, polypeptide, polysaccharide, polysulfone, polypyrrole, polyimidazole, polythiophene, and polyether; epoxies; silica glass; silica gel; titania; siloxane; polyphosphate; hydrogel; agarose; cellulose; and the like.

In some embodiments, the outer layer comprises at least one type of silane. Silane conjugation may be carried out with various types of silanes, including those having trimethoxy silyl, methoxy silyl, or silanol groups at one end, which may be hydrolyzed in basic medium to form a silica shell around the nanoparticle. The silanes may also comprise organic functional groups, examples of which include phosphate and phosphonate groups, amine groups, thiol groups, carbonyl groups (e.g., carboxylic acids, and the like), C₁-C₂₀ alkyl, C₁-C₂₀ alkene, C₁-C₂₀ alkyne, azido groups, epoxy groups, or other functional groups described herein. These functional groups may be bound to the functionalized silanes prior to or subsequent to silane conjugation to the nanoparticle, using methods known in the art.

The outer layer of a luminescent article may also be synthesized using methods known in the art. For example, a luminescent article may first be reacted with a functionalized silane in the presence of a controlled amount of base such that the functionalized silane undergoes substantially only a single hydrolysis reaction, forming a covalent bond with the luminescent article. The degree and rate of silane conjugation can be controlled by varying the temperature and the amount of base in the reaction system. In some embodiments where a hydroxide base is used, the ratio of functionalized silane to base is about 1:1. In other embodiments where a non-hydroxide base is used, the ratio of functionalized silane to base can be less than 1. In some embodiments, a dry of anhydrous organic solvent and a base soluble in the organic solvent are used. The intermediate isolated from the first step may then be suspended in a solvent where it is then reacted with an excess of a base to complete the intraparticle silanization of the functionalized silane moieties. Examples of suitable bases include hydroxide bases, such as tetra-methyl ammonium hydroxide, tetra-butyl ammonium hydroxide, or sodium hydroxide, and non-hydroxide bases such as an alkyl amine. Examples of suitable organic solvents include organic alcohols, hydrocarbons, and benzene derivatives. Specific examples of suitable organic solvents include toluene, cyclohexane, methanol, ethanol, mixtures of ethanol and toluene, DMSO, DMF, and liquid ammonia. In some cases, the silanated luminescent article may precipitate in the organic solvent such that that unreacted silane molecules can be removed. In some cases, toluene is a preferred solvent.

In some embodiments, a reverse microemulsion process may be used to form the outer layer. A “reverse emulsion” or “aqueous in non-aqueous emulsion” is a dispersion of discrete areas of aqueous solvent (aqueous phase) within a non-aqueous solvent. The reverse microemulsion can be produced using a variety of non-polar solvents. In some cases, the non-polar solvent is a hydrocarbon and may be an aliphatic hydrocarbon and, in some cases, is a non-aromatic cyclic hydrocarbon such as cyclopentane, cyclohexane or cycloheptane. In some embodiments, a surfactant (e.g., ionic or non-ionic) may be added to the reverse microemulsion. A “surfactant” is a material exhibiting amphiphilic properties and is used herein as it is commonly used in the art, e.g., for introducing hydrophobic species to hydrophilic environments. Examples of surfactants suitable for use in the present invention include, for example, polyphenyl ethers, such as IGEPAL CO-520, dioctyl sulfosuccinate sodium salt (AOT), trioctyl phosphine oxide (TOPO), and the like.

In some embodiments, the luminescent article comprises a luminescent core. The term “luminescent” is known in the art and refers to the ability to emit electromagnetic radiation (e.g., light). Luminescence may result when a system undergoes a transition from an excited state to a lower energy state, with a corresponding release of energy in the form of a photon. These energy states can be electronic, vibrational, rotational, or any combination thereof. The transition responsible for luminescence can be stimulated through the release of energy stored in the system chemically, kinetically, or added to the system from an external source. The external source of energy can be of a variety of types including chemical, thermal, electrical, magnetic, electromagnetic, or physical, or any other type of energy source capable of causing a system to be excited into a state higher in energy than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy X-ray radiation. Typically, luminescence refers to electromagnetic radiation in the range from UV to IR radiation, and may often refer to visible electromagnetic radiation (i.e., light).

The luminescent core may comprise any material capable of having a luminescence, such as semiconductor nanocrystals, organic dyes, polymers, other organic or inorganic luminescent materials, and the like. In some embodiments, the luminescent core may comprise an organic molecule, such as a fluorescent dye. Using methods known in the art, the fluorescent dye may be covalently bonded to, for example, a silica precursor and condensed to form the luminescent core. A protective, outer layer may then be formed to encapsulate the luminescent core. In one embodiment, the luminescent core may be treated with silica sol-gel monomers to form the outer layer. Examples of fluorescent dyes include, but are not limited to, fluorescein, coumarin, rhodamine, acridine, cyanine, aryl (e.g., pyrene, anthracene, and naphthalene), or heteroaryl moiety, or substituted derivatives thereof. Specific examples of suitable fluorescent dyes include Texas Red, Rhodamine Red, Oregon Green 514, fluorescein-based dyes, as well as other fluorescent dyes found in the Molecular Probes Catalog, 6th Ed., Richard Haugland, Ed., which is incorporated by reference in its entirety. The fluorescent dyes may be at least partially encapsulated by a protective, outer layer, as described herein. The protective outer layer may comprise an inorganic or organic material, and may be tailored to suit a particular application.

In some embodiments, the luminescent core comprises a semiconductor nanocrystal. The semiconductor nanocrystal or quantum dot (e.g., in the luminescent core) may have any suitable semiconductor material composition. For example, semiconductor nanocrystal may be formed of Group II-VI compounds such as semiconductors. The semiconductor materials may be, for example, a Group II-VI compound, a Group III-V compound, or a Group IV element. Suitable elements from Group II of the Periodic Table may include zinc, cadmium, or mercury. Suitable elements from Group III may include, for example, gallium or indium. Elements from Group IV that may be used in semiconductor materials may include, for example, silicon, germanium, or lead. Suitable elements from Group V that may be used in semiconductor materials may include, for example, nitrogen, phosphorous, arsenic, or antimony. Appropriate elements from Group VI may include, for example, sulfur, selenium, or tellurium. In other embodiments, a quantum dot may be comprised of (a) a first element selected from Groups 2, 12, 13 or 14 of the Periodic Table of the Elements and a second element selected from Group 16 of the Periodic Table of the Elements, (b) a first element selected from Group 13 of the Periodic Table of the Elements and a second element selected from Group 15 of the Periodic Table of the Elements, or (c) a Group 14 element. Examples of materials suitable for use in the semiconductive core include, but are not limited to, MgO, MgS, MgSe, MgTe, CaS, CaSe, CaTe, SrS, SrSe, SrTe, BaS, BaSe, BaTe, ZnO, ZnS, ZnSe, ZnTe, CdO, CdS, CdSe, CdTe, HgO, HgS, HgSe, HgTe, AlN, AlP, AlAs, AlSb, Al₂S₃, Al₂Se₃, Al₂Te₃, Ga₂S₃, Ga₂Se₃, GaTe, In₂S₃, In₂Se₃, InTe, SnS, SnSe, SnTe, PbS, PbSe, PbTe, AlP, AlAs, AlSb, GaN, GaP, GaAs, GaSb, InN, InP, InAs, InSb, TiN, TiP, TiAs, TiSb, BP, Si, and Ge, and ternary and quaternary mixtures, compounds, alloys, mixtures, and solid solutions thereof. The semiconductor material may include alloys or mixtures of these materials, or different Groups may be combined together, for example, AlGaAs, InGaAs, InGaP, AlGaAs, AlGaAsP, InGaAlP, or InGaAsP. In some embodiments, the semiconductor material is CdSe, CdTe, ZnSe, or ZnO. In some embodiments, the semiconductor material is ZnO. The specific composition may be selected, in part, to provide the desired optical properties.

The semiconductor nanocrystals may have particle sizes of less than 100 nanometers. In some cases, the average particle size of the semiconductor nanocrystal is less than 20 nanometers; in other cases, the average particle size is less than 5 nanometers (e.g., about 3.5 nanometers). In some embodiments, the average particle size of the quantum dots is greater than 0.5 nanometer. The average particle size may be determined using standard techniques, for example, by measuring the size of a representative number of particles using microscopy techniques (e.g., TEM). It should be understood that the composites may include semiconductor nanocrystal s having different particle sizes which have different light emitting properties.

In some embodiments, the luminescent article may comprise a first material (e.g., luminescent core) having a first lattice structure surrounded by a second material (e.g., outer layer) having a second lattice structure, forming an interfacial region where the first material contacts the second material. The luminescent article may also comprise an additive that may be present in the interfacial region alone or may be present in both the interfacial region and the outer layer or may be present in the luminescent core, the interfacial region, and the outer layer. Alternatively, the additive might not be incorporated into the luminescent article at all, but merely facilitate overgrowth of a high-quality thick outer layer on a semiconductive core. When present in the outer layer, the additive may be uniformly distributed throughout the outer layer or may be distributed as a gradient, i.e., as a gradient that exhibits a decreasing concentration in an outward direction from the semiconductive core. The interfacial region may be discontinuous, comprise a monolayer, or comprise many monolayers, and the region may incorporate several combinations of elements, including elements not native to either the core or shell structures. For example, oxygen atoms may be introduced into the interfacial region during synthesis. Other elements that may be used as an additives include, but are not limited to, Group 2, 12, 13, 14, 15 and 16 elements, such as Fe, Nb, Cr, Mn, Co, Cu, and Ni.

The emission wavelength of a semiconductor nanocrystal may be governed by the size of the nanocrystal. These emissions may be controlled by varying the particle size or composition of the particle. The light emitted by a semiconductor nanocrystals may have very narrow wavelengths, for example, spanning less than about 100 nm, preferably less than about 80 nm, more preferably less than about 60 nm, more preferably less than about 40 nm, and more preferably less than about 20 nm. The semiconductor nanocrystal may emit a characteristic emission spectrum which can be observed and measured, for example, spectroscopically. Thus, in certain cases, many different semiconductor nanocrystals may be used simultaneously, without significant overlap of the emitted signals. The emission spectra of a semiconductor nanocrystal may be symmetric or nearly so. Unlike some fluorescent molecules, the excitation wavelength of the semiconductor nanocrystal may have a broad range of frequencies. Thus, a single excitation wavelength, for example, a wavelength corresponding to the “blue” region or the “purple” region of the visible spectrum, may be used to simultaneously excite a population of nanocrystals, each of which may have a different emission wavelength. For example, a cadmium selenide crystal of 3 nanometers may produce a 520 nanometer emission, while a cadmium selenide crystal of 5.5 nanometers in diameter may produce a 630 nanometer emission upon excitation with light having a frequency of 450 nanometers, corresponding to “blue” light. Multiple signals, corresponding to, for example, multiple chemical or biological assays, may thus be simultaneously detected and recorded.

The quantum dots or semiconductor nanocrystals may be synthesized by methods known in the art, such as flocculation with a non-solvent (e.g., methanol). Optionally, the semiconductor nanocrystals thus prepared and isolated maybe subjected to an amine-treatment step prior to formation of the outer layer. In one embodiment, the semiconductor nanocrystals are prepared by injecting first and second precursors into a reaction solution held at a temperature sufficient to induce homogeneous nucleation of discrete semiconductor nanocrystals. Once the monodisperse particle population containing the individual semiconductive cores has been formed, the semiconductive cores may be isolated from the first solvent and then placed in a second solvent to form a core solution. Alternatively, the core solution can simply be comprised of the original solution in which the monodisperse population of cores is formed. Using this method, the luminescent luminescent articles can be formed in a “one pot” synthesis (e.g., in a single reaction vessel). In the “one pot” method, any unreacted precursors from the semiconductive core synthesis can be used as the additive material during formation of the outer layer.

The temperature at which the outer layer is formed on the semiconductive core is related to the quality of the resultant nanoparticle. Outer layer formation at relatively higher temperatures may cause the individual cores to begin to grow via Ostwald ripening, with resulting deterioration of the size distribution of the particles, leading to broader spectral line widths. Formation of the outer layer at relatively low temperatures could lead to incomplete decomposition of the precursors or to reduced integrity of the lattice structure of the outer layer. Typical temperatures for forming the outer layer range from about 100° C. to about 300° C. The actual temperature range may vary, depending upon the relative stability of the precursors and the semiconductive core.

Following nucleation, the nanocrystals may be allowed to grow until reaching the desired size and then quenched by dropping the reaction temperature. Particle size and particle size distribution during the growth stage of the core reaction may be approximated by monitoring the absorption or emission peak positions and line widths of the samples. Dynamic modification of reaction parameters such as temperature and monomer concentration in response to changes in the spectra may allow the tuning of these characteristics. Cores thus prepared can be isolated using methods well known to those skilled in the art, such as flocculation with a non-solvent (e.g., methanol). Optionally, the cores thus prepared and isolated may be subjected to an amine-treatment step prior to outer layer formation. Such amine treatments are disclosed by Talapin et al., Nano Letters 2001, 1, 207, incorporated herein by reference, and will be well understood by those of skill in the art. The concentrations of the additive precursor and the core and outer layer precursors, and the rate of the addition of these precursors to the core solution, are selected to promote heterogeneous growth of the outer layer onto the semiconductive core rather than homogeneous nucleation, to produce semiconductive cores comprised of elements of the first and second outer layer precursors. Conditions favoring heterogeneous growth include dropwise addition, e.g., 1-2 drops/second, of solutions containing the first and second outer layer precursors to the core solution, and maintenance of the precursors at low concentrations. Low concentrations typically range from 0.0005-0.5 M. In this manner, a outer layer may be formed over the semiconductive core with an interfacial region formed between the semiconductive core and outer layer.

Suitable solvents may be selected from the group consisting of acids (particularly fatty acids), amines, phosphines, phosphine oxides, phosphonic acids (and phosphoramides, phosphates, phosphates, etc.), and mixtures thereof. Other solvents, including alkanes, alkenes, halo-alkanes, ethers, alcohols, ketones, esters, and the like, are also useful in this regard, particularly in the presence of added luminescent article ligands. It is to be understood that the first and second solvents may be the same and, in “one pot”-type synthesis, may comprise the same solution.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc.

As used herein in the specification and in the claims, unless clearly indicated to the contrary, “or” should be understood to have the same meaning as “and/or” as defined above. For example, when separating items in a list, “or” and “and/or” each shall be interpreted as being inclusive, i.e., the inclusion of at least one, but also including more than one, of a number or list of elements, and, optionally, additional unlisted items. In general, the term “or” as used herein shall only be interpreted as indicating exclusive alternatives (i.e. “one or the other but not both”) when preceded by terms of exclusivity, such as “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

General Methods. All the chemicals, if not specified, were purchased from commercial sources (Sigma-Aldrich, Lancaster, Alfa Aesar and Gelest), and used without further purification. The ZnO nanocrystals were synthesized using methods known in the art (e.g., Meulenkamp, E. A., J. Phys. Chem. B 1998, 102, 5566, incorporated herein by reference). CdSe quantum dots were prepared using a technique described in Peng et. al., J. Am. Chem Soc. 2001, 123, 183. Absorption spectra of samples were measured at room temperature on an Agilent 8453 UV-Vis spectrometer. Luminescence spectra were measured at room temperature on a Jobin Yvon Horiba Fluorolog luminescence spectrometer.

### Example 1

### Synthesis of NH₂—ZnO Nanocrystals

Approximately 30 mg of the NH₂—ZnO nanocrystal was dissolved in 10 mL of deionized water. The stock solution was then filtered with a 0.2-μm membrane syringe filter immediately prior to use. The concentration of NH₂—ZnO nanocrystals solutions was quantified by UV-visible spectrometry at a wavelength of 330 nm.

The solution of NH₂—ZnO nanocrystals may be diluted to the desired concentration immediately prior to the experiment. In some cases, the solution of NH₂—ZnO nanocrystals may be most stable at ˜3 mg/mL.

### Example 2

### High Throughput Screening of Aldehydes

Various aldehydes samples were each dissolved in dimethyl sulfoxide (DMSO). Each aldehyde sample solution (75 μL) was combined with an aqueous solution of NH₂—ZnO nanocrystals (5 μg/mL, 75 μL) in an individual well of a 96-well plate. The plate was then exposed to UV irradiation (λₘₐₓ=365 nm, 50 W) for two minutes from a flat-panel transilluminator (Wealtec). The luminescence intensity at 545 nm (excited at 345 nm) was recorded by a microplate reader (Tecan).

### Example 3

### Photostability of NH₂—ZnO Nanocrystals

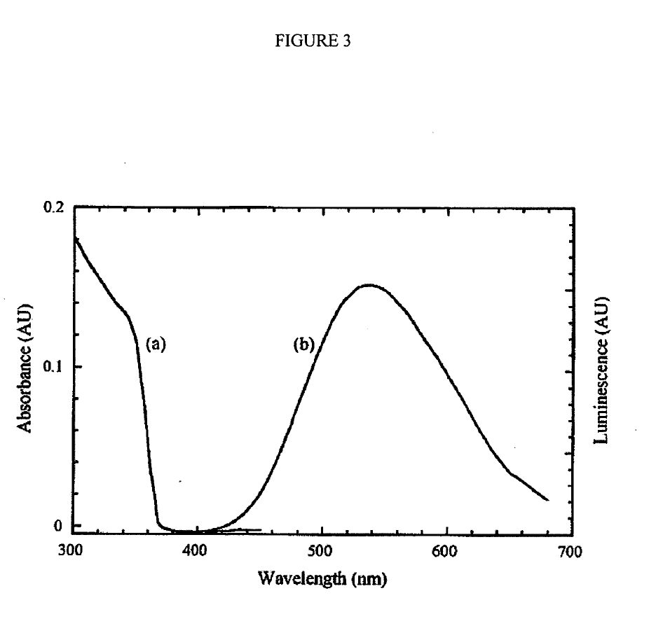

Upon exposure to UV irradiation, NH₂—ZnO nanocrystals displayed broad absorption that dropped sharply above 350 nm and an emission peak at 545 nm (FIG. 3). The luminescence of the ZnO nanocrystals was attributed to the “surface trap effect,” yielding a relatively broad emission peak (120 nm in bandwidth). The amine concentration was estimated to be 2.2 mM for the stock NH₂—ZnO nanocrystal solution (3 mg/mL) by fluorescamine titration.

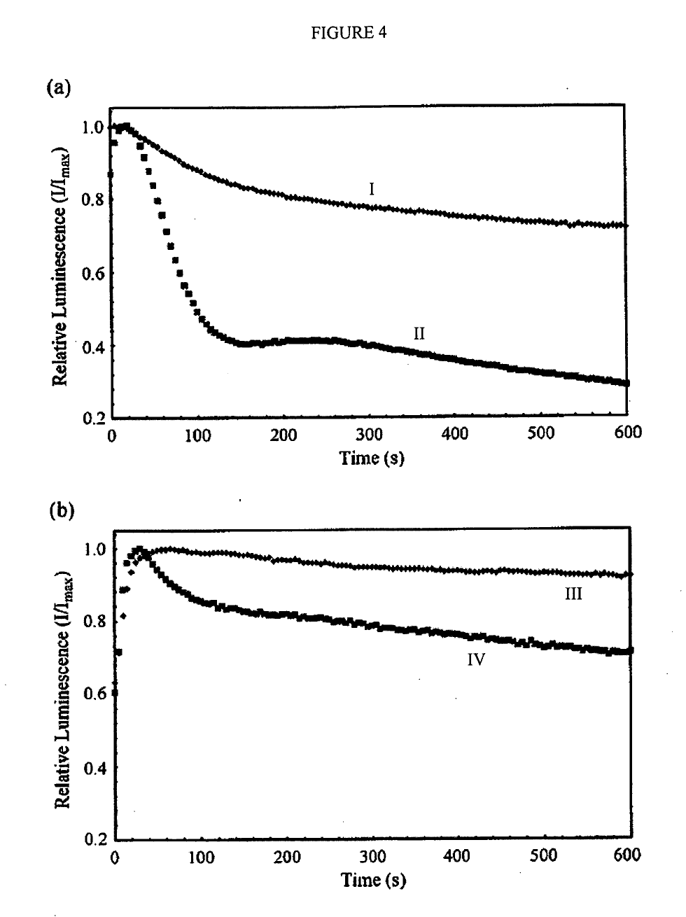

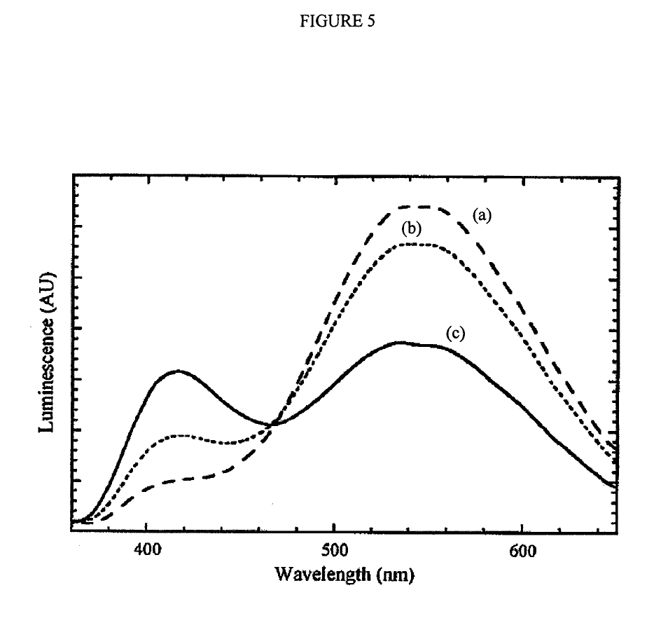

The photostability of the NH₂—ZnO nanocrystals was measured by the kinetic luminescence. FIG. 4 shows the kinetic luminescence of a 0.03 mg/mL NH₂—ZnO nanocrystal solution in the absence and presence of 0.5 mM o-phthaldehyde. The luminescence was measured in water (FIG. 4A) and 5 mM borate buffer solution (pH=8.9) (FIG. 4B), with an excitation wavelength of 345 nm and an emission wavelength of 545 nm. A diluted NH₂—ZnO nanocrystal solution (0.03 mg/mL) was continuously excited at a wavelength of 345 nm, and the emission intensity at 545 nm was recorded every 5 seconds.

FIG. 4A shows the luminescence intensity in the absence (I) and presence (II) of 0.5 mM o-phthaldehyde in water. In the absence of aldehyde, a 29% reduction in luminescence intensity was observed after 10 min of exposure to UV irradiation. The luminescence intensity was further reduced upon extended exposure. FIG. 4B shows the luminescence intensity in the absence (III) and presence (IV) of 0.5 mM o-phthaldehyde after exposure to UV irradiation in the presence of 5 mM borate buffer (pH=8.9). The presence of the buffer stabilized the NH₂—ZnO nanocrystals, and less than an 8% reduction in luminescence intensity was observed after 10 min of exposure to UV irradiation. A 60% luminescence intensity increase was observed for the NH₂—ZnO nanocrystals in borate buffer solution, compared to NH₂—ZnO nanocrystals in aqueous solution.

### Example 4

### Photostability of NH₂—ZnO Nanocrystals in the Presence of o-Phthaldehyde

In the presence of 0.5 mM of o-phthaldehyde, the surface amine groups of NH₂—ZnO nanocrystals may react reversibly with o-phthaldehyde to form imines. Upon exposure to UV irradiation, a greater reduction in luminescence intensity may be observed (71% and 30% in aqueous and borate buffer solutions, respectively) after 10 min of UV exposure. Without UV exposure, the luminescence of the aqueous nanocrystals solution was completely quenched after 1 day under normal daylight, suggesting that the imine-functionalized ZnO nanocrystals may be more susceptible to photobleaching. Similar experiments were conducted with different o-phthaldehyde concentrations. FIG. 5 shows the emission spectra of NH₂—ZnO nanocrystals solution (0.03 mg/mL) in the presence of o-phthaldehyde solutions of (a) 0.05 mM, (b) 0.125 mM, and (c) 0.25 mM after the kinetic luminescence experiment in FIG. 4. The emission spectra showed a positive relationship between the reduction of peak intensity at 545 nm and the concentration.

A new emission peak at 419 nm emerged from the phenylimine luminophore. This peak intensity was positively related to the o-phthaldehyde concentration. However, a blue shift of this emission occurred when the o-phthaldehyde concentration was much greater than the corresponding amino group concentration. This could be due to imine group oxidation or formation of aggregates.

### Example 5

### Size of NH₂—ZnO Nanocrystals

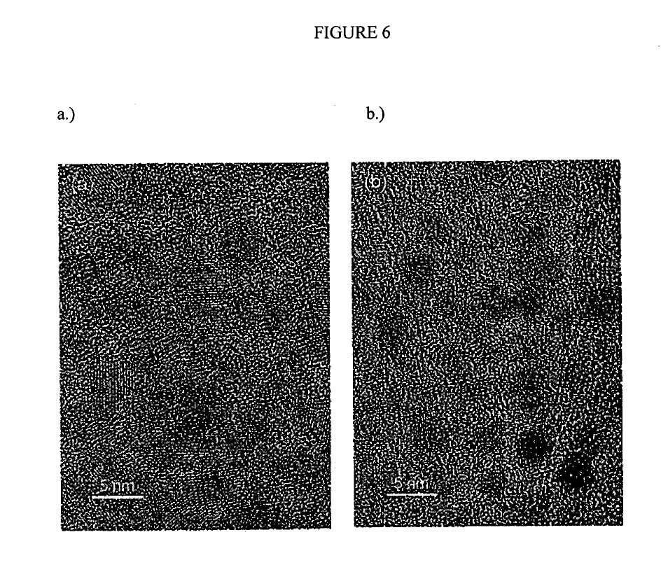

FIG. 6 shows TEM images of NH₂—ZnO nanocrystals (a) before and (b) after treatment with 0.5 mM of o-phthaldehyde under exposure to UV light for 10 minutes. The original size of the nanocrystals was ˜4-5 nm based on transmission electron microscopy (TEM) (FIG. 6A). The crystallinity of ZnO was clearly observed. However, the thin silane coating could not be distinguished by TEM. Upon treatment of the NH₂—ZnO nanocrystals with o-phthaldehyde, the grain size of the nanocrystals was reduced to 2-3 nm after UV exposure for 10 min (FIG. 6B), indicating that photobleaching of the nanocrystals may have resulted from increasing photodissolution of ZnO nanocrystals upon reaction of the surface amine groups with o-phthaldehyde. The increasing photosolubility may indicate the lower affinity between the imine groups and nanocrystals, compared to that between the amine groups and nanocrystals, leading to a more porous shell and, thus, rendering the core nanocrystals more susceptible to photodissolution.

### Example 6

### Detection of Aldehydes

To develop the “turn-off” photobleaching response towards aldehyde detection, a similar protocol as previously described in Example 2 was employed. Aldehyde and control compounds (9 mM) were dissolved in DMSO due to their limited solubility in water, and the samples were each placed in individual wells of a multi-well microplate. After mixing each aldehyde sample with an equal volume of aqueous solution of NH₂—ZnO nanocrystals (5 μg/mL), the microplate was exposed to UV light from a flat-panel UV transilluminator ((λₘₐₓ=365 nm). The luminescence intensity at 545 nm (excited at 345 nm) was recorded after 2 min of UV exposure. This set-up enabled us to directly monitor the compatibility of the proposed detection method with high-throughput screening technology.

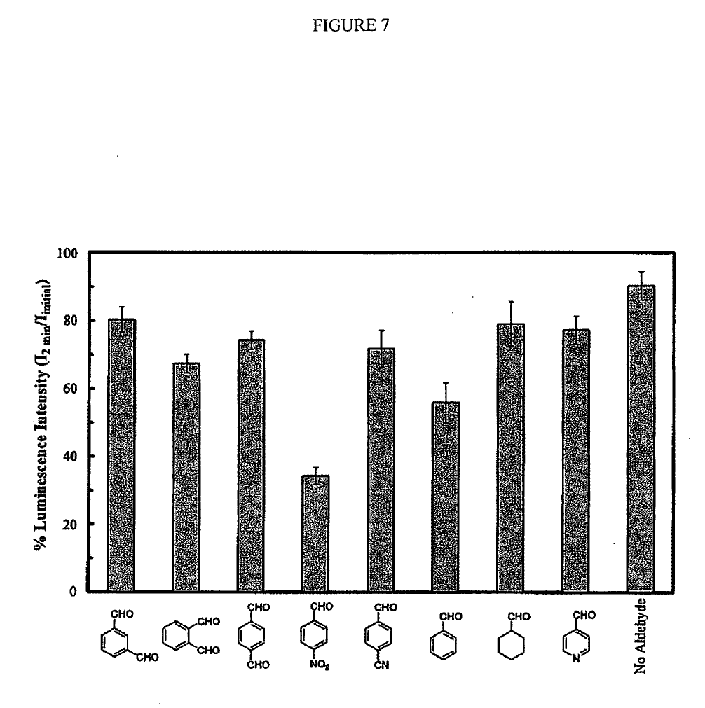

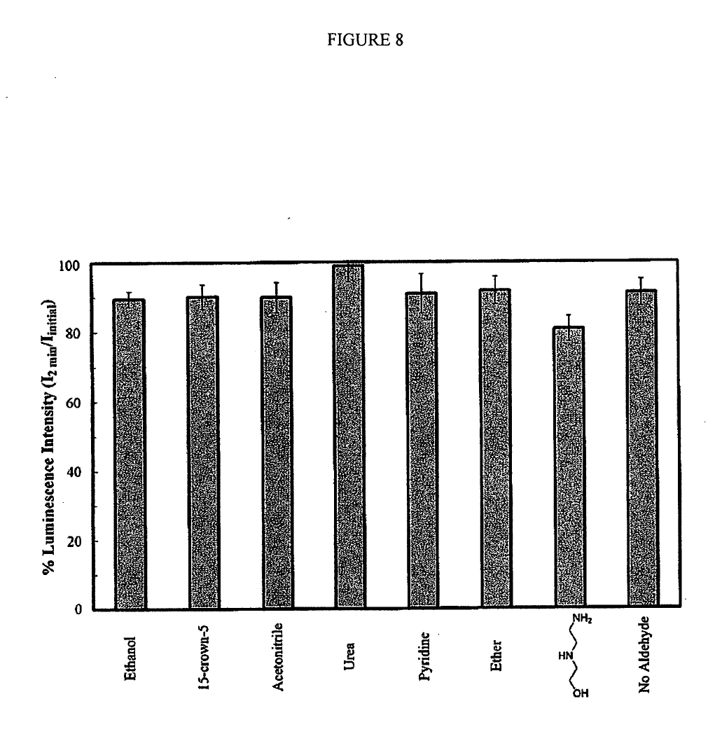

FIG. 7 shows the percentage of luminescence intensity of NH₂—ZnO nanocrystals (5 μg/mL) in the presence of different aldehydes (0.9 mM) after two minutes of exposure to UV irradiation. As shown in FIG. 7, the presence of various aldehydes generally quenched the luminescence of NH₂—ZnO nanocrystals by 20-70%, whereas only 10% quenching was noted in the control after 2 min of UV exposure. It is noteworthy that even the aliphatic aldehydes responded similarly to the aromatic aldehydes under the designed protocol. The luminescence intensity was reduced by 66% in the presence of p-nitrobenzaldehyde. The quenching of luminescence was reduced compared to the earlier kinetic experiments. This was partly due to the much lower absorption coefficient of NH₂—ZnO nanocrystals at the wavelength of 365 nm, and partly due to the reduced UV exposure time. Photostability experiments with organic compounds with different functional groups all showed only minor reduction in luminescence intensity (see FIG. 8), as with the control experiment. FIG. 8 shows the luminescence response of NH₂—ZnO nanocrystals solution (5 μg/mL) to various control organic compounds (0.9 mM) after two minutes of exposure to UV irradiation. Only 2-amine-ethylamineethanol resulted in ˜10% more intensity drop than the control experiment.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, materials, reaction conditions, and configurations described herein are meant to be exemplary and that actual parameters, materials, reaction conditions, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood, unless otherwise indicated, to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently ““at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed is:

1. A method for determining an analyte via interaction of the analyte with a: luminescent article, comprising:
providing a sample suspected of containing an analyte;
exposing the sample to a luminescent article comprising an outer layer and, if the analyte is present, allowing the analyte to become immobilized with respect to the article via interaction between the analyte and the outer layer, wherein the outer layer is modified by said interaction;
determining a first emission of the luminescent article;
exposing the nanoparticle to electromagnetic radiation for a period of time and under conditions sufficient to cause a change in a luminescence characteristic of the nanoparticle;
determining a second emission of the luminescent article; and
determining a variance between the first emission and the second emission indicative of the presence of the analyte,
wherein modification of the outer layer increases the susceptibility of the article to a change in the luminescence characteristic upon exposure of the article to the electromagnetic radiation for the period of time and under the conditions, such that, in the absence of the analyte, exposure of the luminescent article to the electromagnetic radiation for the period of time and under the conditions does not result in said variance between the first and second emissions.

2. A method as in claim 1, wherein, in the absence of the analyte, exposure of the luminescent article to the electromagnetic radiation for the period of time and under the conditions results in a different variance between the first and second emissions.

3. A method as in claim 1, wherein the variance in the absence of the analyte is smaller than the variance in the presence of the analyte.

4. A method as in claim 1,
wherein the outer layer of the luminescent article comprises a plurality of functional groups having an affinity for a surface of the article, and immobilization of the analyte with respect to the article causes the functional groups to become increased in separation from the surface of the article, increasing the susceptibility of the article to the change in the luminescence characteristic upon exposure of the article to the electromagnetic radiation for the period of time and under the conditions.

5. A method as in claim 1, wherein the outer layer is a self-assembled, tightly-packed structure and, in the presence of the analyte, the outer layer interacts with the analyte to disrupt the self-assembled, tightly-packed structure, increasing the susceptibility of the article to the change in the luminescence characteristic upon exposure of the article to the electromagnetic radiation for the period of time and under the conditions.

6. A method as in claim 1, wherein the outer layer comprises at least one type of silane.

7. A method as in claim 1, wherein the luminescent article comprises a semiconductor nanocrystal.

8. A method as in claim 7, wherein the semiconductor nanocrystal is MgO, MgS, MgSe, MgTe, CaS, CaSe, CaTe, SrS, SrSe, SrTe, BaS, BaSe, BaTe, ZnO, ZnS, ZnSe, ZnTe, CdO, CdS, CdSe, CdTe, HgO, HgS, HgSe, HgTe, AlN, AlP, AlAs, AlSb, Al₂S₃, Al₂Se₃, Al₂Te₃, Ga₂S₃, Ga₂Se₃, GaTe, In₂S₃, In₂Se₃, InTe, SnS, SnSe, SnTe, PbS, PbSe, PbTe, AlP, AlAs, AlSb, GaN, GaP, GaAs, GaSb, InN, InP, InAs, InSb, TiN, TiP, TiAs, TiSb, BP, Si, Ge, alloys thereof, such as AlGaAs, InGaAs, InGaP, AlGaAs, AlGaAsP, InGaAlP, or InGaAsP, ternary or quaternary mixtures thereof, compounds thereof, or solid solutions thereof.

9. A method as in claim 7, wherein the semiconductor nanocrystal is CdSe, CdTe, ZnSe, and/or ZnO.

10. A method as in claim 7, wherein the luminescent article comprises ZnO.

11. A method as in claim 1, wherein the outer layer comprises an amine, a thiol, a carboxylic acid, an anhydride, and/or an alcohol.

12. A method as in claim 11, wherein the outer layer comprises an amine.

13. A method as in claim 1, wherein the interaction comprises forming a covalent bond, an ionic bond, a hydrogen bond, and/or Van der Waal interactions with the analyte.

14. A method as in claim 1, wherein the interaction comprises forming a covalent bond with the analyte.

15. A method as in claim 1, wherein the analyte comprises an aldehyde.

16. A method as in claim 1, wherein the analyte is a biological molecule.

17. A method as in claim 1, wherein the luminescent article comprises a fluorescent dye.

18. A method for determining an analyte, comprising:
exposing a luminescent article to electromagnetic radiation in the presence of a sample suspected of containing an analyte, wherein the analyte affects a change in a luminescence characteristic of the article responsive to the electromagnetic radiation; and
if the analyte is present, determining the analyte by determining a change in the luminescence characteristic of the article resulting from said exposure to electromagnetic radiation.

19. A method as in claim 18, wherein the exposing comprises exposing the luminescent article to electromagnetic radiation for a period of time and under conditions sufficient to cause a change in a luminescence characteristic of the nanoparticle.

20. A method as in claim 19, wherein the change in the luminescence characteristic in the absence of the analyte is different from the change in the luminescence characteristic in the presence of the analyte.

21. A method as in claim 19, wherein the change in the luminescence characteristic in the absence of the analyte is smaller in magnitude than the change in the luminescence characteristic in the presence of the analyte.

22. A method as in claim 18,
wherein the luminescent article comprises an outer layer comprising a plurality of functional groups having an affinity for a surface of the article, and immobilization of the analyte with respect to the article causes the functional groups to become increased in separation from the surface of the article, increasing the susceptibility of the article to the change in the luminescence characteristic upon exposure of the article to the electromagnetic radiation for the period of time and under the conditions.

23. A method as in claim 22, wherein the outer layer comprises at least one type of silane.

24. A method as in claim 18, wherein the outer layer is a self-assembled, tightly-packed structure and, in the presence of the analyte, the outer layer interacts with the analyte to disrupt the self-assembled, tightly-packed structure, increasing the susceptibility of the article to the change in the luminescence characteristic upon exposure of the article to the electromagnetic radiation for the period of time and under the conditions.

25. A method as in claim 24, wherein the outer layer comprises at least one type of silane.

26. A method as in claim B, wherein the luminescent article comprises a semiconductor nanocrystal.

27. A method as in claim 26, wherein the semiconductor nanocrystal is MgO, MgS, MgSe, MgTe, CaS, CaSe, CaTe, SrS, SrSe, SrTe, BaS, BaSe, BaTe, ZnO, ZnS, ZnSe, ZnTe, CdO, CdS, CdSe, CdTe, HgO, HgS, HgSe, HgTe, BlN, BlP, BlBs, BlSb, Bl₂S₃, Bl₂Se₃, Bl₂Te₃, Ga₂S₃, Ga₂Se₃, GaTe, In₂S₃, In₂Se₃, InTe, SnS, SnSe, SnTe, PbS, PbSe, PbTe, BlP, BlBs, BlSb, GaN, GaP, GaBs, GaSb, InN, InP, InBs, InSb, TiN, TiP, TiBs, TiSb, BP, Si, Ge, alloys thereof, such as BlGaBs, InGaBs, InGaP, BlGaBs, BlGaBsP, InGaBlP, or InGaBsP, ternary or quaternary mixtures thereof, compounds thereof, or solid solutions thereof.

28. A method as in claim 26, wherein the semiconductor nanocrystal is CdSe, CdTe, ZnSe, and/or ZnO.

29. A method as in claim 26, wherein the luminescent article comprises ZnO.

30. A method as in claim B, wherein the outer layer comprises an amine, a thiol, a carboxylic acid, an anhydride, and/or an alcohol.

31. A method as in claim 30, wherein the outer layer comprises an amine.

32. A method as in claim 18, wherein the interaction comprises forming a covalent bond with an analyte.

33. A method as in claim 18, wherein article and the analyte have an interaction comprising forming a covalent bond, an ionic bond, a hydrogen bond, and/or Van der Waal interactions with the analyte.

34. A method as in claim 33, wherein the interaction comprises forming a covalent bond with the analyte.

35. A method as in claim 18, wherein the analyte comprises an aldehyde.

36. A method as in claim 18, wherein the analyte is a biological molecule.

37. A method as in claim 18, wherein the luminescent article comprises a fluorescent dye.
